(19) United States
(12) Patent Application Publication
(10) Pub. No.: US 2017/0231581 A1
(43) Pub. Date: Aug. 17, 2017

(54) CT IMAGE RECONSTRUCTION METHOD, CT IMAGE RECONSTRUCTION DEVICE, AND CT SYSTEM

(71) Applicant: HITACHI, LTD., Tokyo (JP)

(72) Inventors: Xingdong SHENG, Beijing (CN); Yingjie HAN, Beijing (CN); Taiga GOTO, Tokyo (JP); Keisuke YAMAKAWA, Tokyo (JP)

(21) Appl. No.: 15/503,041

(22) PCT Filed: Jun. 15, 2015
(86) PCT No.: PCT/JP2015/067154

§ 371 Date: Feb. 10, 2017

(30) Foreign Application Priority Data
Sep. 16, 2014 (CN) 201410472082.X

(51) Int. Cl. A61B 6/03; G06T 7/70; G06T 11/00

(52) U.S. Cl. CPC A61B 6/032; G06T 11/003; G06T 7/70; G06T 2207/10081; G06T 2207/30004

(57) ABSTRACT

A CT image reconstructing method, a CT image reconstructing device and a CT system are provided for reducing motion artifacts in CT images in case of motion of an object. The CT image reconstructing method reconstructs CT images from projection data obtained by X-ray scanning, including a moving object position detecting step for detecting a position of a moving object in a CT image; a partial angle selecting step for selecting a view point and an angle range according to said position of the moving object and selecting data of partial angles in said projection data according to said view point and said angle range; a partial angle constraint step for generating partial angles constraint conditions according to the data of said partial angles; and an iterative reconstruction step for generating CT images by iterative reconstruction, thereby improving temporal resolution of CT images of moving objects and reducing motion artifacts.

### TECHNICAL FIELD

The present invention relates to a CT image reconstructing method, a CT image reconstructing device and a CT system, particularly to a CT image reconstructing method, a CT image reconstructing device and a CT system in case of motions of an object.

### BACKGROUND

Nowdays, X-ray Computerized tomography (CT) technology is more and more widely used. For example, CT technology is widely used for many fields such as scientific research, organism data acquisition, and body inspection. Among them, CT images have been used as for example intermediate data to diagnose diseases for 30 years. The investigation of CT image reconstructing approaches to reduce radiation dose, improve CT image quality and reduce image artifacts has always been a focus in the study.

In practical applications, CT image reconstructing approaches mainly include filtered back projection and iterative reconstruction. Filtered back projection is a traditional approach for CT image reconstruction and has been widely applied in present CT products. However, in filtered back projection, the projection data for reconstructing images is assumed to be noise-free. While in fact, noise is always accompanying projection data, particularly in case of low dose scanning. Therefore, it is difficult to obtain high-quality CT images by filtered back projection.

However, with the development of application fields of CT technology, the width and depth of CT applications have achieved an unprecedented height. Under such new background, the industry has proposed new and higher requirements for the safety consideration and image quality of CT applications. This makes the filtered back projection difficult to meet new requirements.

In view of the above new requirements, in high-end applications, iterative reconstruction has been paid more attention and investigated. Iterative reconstruction can handle image artifacts caused by electronic noise and other physical factors well, thereby reducing X-ray dose upon scanning while ensuring image quality. However, its huge computational complexity leads to slow imaging speed and hence the difficulty of practical application. In recent years, with the rapid development of computer hardware and computer science, applying iterative reconstruction to practical products has become possible.

The image reconstruction process may be represented by formula 1, wherein M denotes the system matrix of CT, X is the image to be reconstructed, and P is the projection data obtained by CT scanning.

MX=P (Formula 1)

With respect to iterative reconstruction, the final image X is obtained by evaluating the minimization objective function Oₐᵣₜ in formula 2 in an iteration procedure, that is, the reconstructed image X should meet the consistency condition for the projection data obtained by CT scanning.

Oₐᵣₜ=∥MX−P∥₂ (Formula 2)

In recent years, the compress sensing theory has been investigated extensively in the CT image reconstruction field (referring to prior art document 1). According to the compress sensing theory, apriori knowledge is introduced in the iterative reconstruction of CT images as constrain conditions, which can effectively reduce iteration numbers of the iterative reconstruction and improve image reconstruction quality.

The CT image reconstruction process of compress sensing theory may be represented by formula 3. The formula 3 indicates that when the data consistency condition expressed by formula 1 is met, the objective function of the constraint item of the apriori knowledge is minimized, that is, the L1 norm of the image reconstructed under constraint is minimal after sparse transformation. Ψ is the sparse transformation matrix and commonly includes various wavelet transformations.

min∥ΨX∥₁, s.t. MX=P (Formula 3)

In the method of prior art document 1, it has been proved the effectiveness of Ψ being TV (Total Variation) transformation.

The prior art document 2, by introducing a known apriori image as constraint based on prior art document 1, can reconstruct clear images in case of sparse projection data with apriori image constrain added to its constrain items, as shown in formula 4.

min[α∥Ψ₁(X−Xₚ)∥₁+(1−α)∥Ψ₂(X)∥₁], s.t. MX=P (Formula 4)

Wherein Ψ₁, Ψ₂ are both sparse transformation matrices, Xₚ is an apriori basic image estimated with a certain method, and a is the weight. In this manner, even in case of few (sparse) projection data, due to the constraint of the apriori image, it is also possible to reconstruct clear images, thereby effectively reduce radiation dose of X-ray.

As described above, CT technology, especially CT image reconstructing technology, has developed rapidly in recent years. However, in practical CT applications, there may be still many artifacts in case of motion of the object. This is because that in the CT rotation scanning process, moving objects causes inconsistency of CT scanning data, and it is thus difficult to reconstruct clear images with good consistency. For example, in the process of scanning heart, since the heart is beating during CT scanning of a round of rotation, internal structures would change while scanning at each angle. Therefore, motion artifacts would occur when reconstructing images with all angles, which makes it difficult to reconstruct clear images. However, demands for CT scanning moving objects such as hearts are increasing in recent years, and this technical problem is urgent to be investigated and addressed.
Prior art document 1: Sidky E Y, Pan X. Image reconstruction in circular cone-beam computed tomography by constrained, total-variation minimization[J]. Physics in medicine and biology, 2008, 53(17): 4777.
Prior art document 2: Chen G H, Tang J, Leng S. Prior image constrained compressed sensing (PICCS): a method to accurately reconstruct dynamic CT images from highly undersampled projection data sets[J]. Medical physics, 2008, 35(2): 660-663.

### SUMMARY

In view of the above technical problems of the prior art, the present application proposes a CT image reconstructing method, a CT image reconstructing device and a CT system for reducing motion artifacts in CT images in case of motion of an object.

The present invention provides a CT image reconstructing method for reconstructing CT images from projection data obtained by X-ray scanning, characterized by including a moving object position detecting step for detecting a position of a moving object in a CT image; a partial angle selecting step for selecting a view point and an angle range according to said position of the moving object and selecting data of partial angles in said projection data according to said view point and said angle range; a partial angle constraint step for generating a partial angle constraint condition according to the data of the partial angles; and an iterative reconstruction step for generating CT images by iterative reconstruction with the partial angle constraint condition.

With respect to the CT image reconstructing method according to the present invention, the partial angle projection data is introduced into the iterative reconstruction algorithm as the constrain condition for data consistency. Since the projection data of partial angles can limit the inconsistency among images during scanning process, using projection data of partial angles as the constrain condition for the entire restriction result, it is possible to make the iterative reconstruction results proceed toward the direction of meeting consistency, that is, the direction of having less motion artifacts. Therefore, it is possible to improve temporal resolution of CT images of moving objects and reduce motion artifacts.

In the above-described CT image reconstructing method, it is possible that in case that the position of said moving object does not belong to a central region of the CT image, in said partial angle selecting step, a view point on a side different from the position of said moving object with respect to a center of the CT image is selected; and in case that the position of said moving object belongs to a central region of the CT image, in said partial angle selecting step, an arbitrary view point is selected.

It is also possible that in case that the position of said moving object does not belong to the central region of the CT image, in said partial angle selecting step, a view point that is farthest from the position of said moving object is selected.

Thereby, in case that the position of said moving object does not belong to a central region of the CT image, selecting a view point on a side different from the position of said moving object with respect to a center of the CT image, such as the view point farthest from the position of the moving object, can reduce influence on projection data by the motion of moving object and in turn further reduce motion artifacts.

In the above-described CT image reconstructing method, said partial angle selecting step may also select data of a preset angle range centered on said view point as data of said partial angles.

Thereby, by centering on the view point and appropriately setting the angle range according to experimental values, it is possible to further reduce motion artifacts.

In the above-described CT image reconstructing method, said iterative reconstructing step may also generate a CT image by iterative reconstruction with the constraint condition obtained by weighted adding of said partial angle constraint condition and an overall image constraint condition.

Therefore, it is possible to improve temporal resolution of the CT image of the moving object and reduce motion artifacts by introducing partial angle projection data into, for example, iterative reconstruction based on compress sensing theory as a constraint condition for data consistency.

In addition, the present invention further provides a CT image reconstructing device for reconstructing CT images from projection data obtained by X-ray scanning, characterized by including a moving object position detecting unit for detecting a position of a moving object in a CT image; a partial angle selecting unit for selecting a view point and an angle range according to said position of the moving object and selecting data of partial angles in said projection data according to said view point and said angle range; a partial angle constraint unit for generating a partial angle constraint condition according to the data of said partial angles; and an iterative reconstructing unit for generating a CT image by iterative reconstruction with the partial angle constraint condition.

In addition, the present invention further provides a CT system for scanning with X-ray and outputting CT images, characterized by including: a CT scanner for obtaining projection data by scanning with X-ray; a CT image reconstructing device for detecting a position of a moving object in a CT image, selecting a view point and an angle range according to said position of the moving object and selecting data of partial angles in said projection data according to said view point and said angle range, generating a partial angle constraint condition according to the data of said partial angles, and generating a CT image by iterative reconstruction with the partial angle constraint condition; and a CT image output device for outputting the CT image reconstructed by said CT image reconstructing device.

All the above-mentioned various approaches of the CT image reconstructing method according to the present invention may be implemented with the CT image reconstructing device and the CT system. In addition, the present invention is not limited to the above-described CT image reconstructing method, the CT image reconstructing device and the CT system, and may also be implemented by causing a computer to execute the CT image reconstructing program of the above-mentioned CT image reconstructing method or with integrated circuits constituting the above-described CT image reconstructing device.

### BRIEF DESCRIPTION OF THE DRAWINGS

FIG. 1 is a structure block diagram of a CT system having a CT image reconstructing device according to an embodiment of the present invention.

FIG. 2 is a structure block diagram of a CT image reconstructing device according to an embodiment of the present invention.

FIG. 3 is a flow chart of a CT image reconstructing method according to an embodiment of the present invention.

FIG. 4 is a flow chart of a specific example of CT image reconstruction according to an embodiment of the present invention.

FIGS. 5A to 5D are principle diagrams of the effectiveness of partial angle constrains according to an embodiment of the present invention.

FIGS. 6A and 6B are principle diagrams of an approach for detecting positions of a moving object according to a specific example of an embodiment of the present invention, and FIG. 6C is their flow chart.

FIG. 7A is a principle diagram of another approach for detecting positions of a moving object according to a specific example of an embodiment of the present invention, and FIG. 7B is its flow chart.

FIGS. 8A and 8B are principle diagrams of partial angle selection and setting according to a specific example of an embodiment of the present invention, and FIG. 8C is their flow chart.

### DETAILED DESCRIPTION

The present invention will be described in more detail below with respect to accompanying drawings and implementations. Furthermore, the same reference numeral will be assigned to the same or corresponding parts in drawings and repeated description will be omitted.

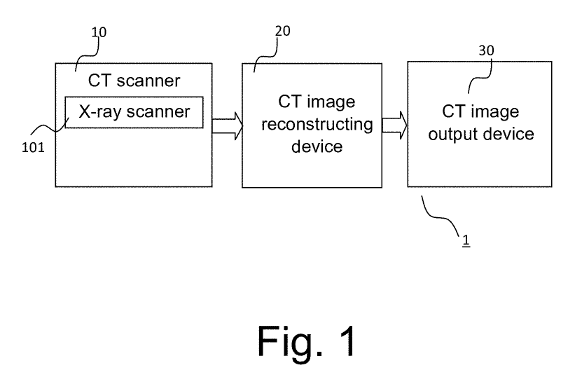

The CT system having the CT image reconstructing device according to the present invention will be described in detail first. FIG. 1 is a structure block diagram of a CT system having a CT image reconstructing device according to an embodiment of the present invention. As shown in FIG. 1, CT system 1 mainly includes a CT scanner 10, a CT image reconstructing device 20 and a CT image output device 30.

The CT scanner 10 performs scanning with X-ray to obtain projection data. The CT scanner 10 includes an X-ray scanner 101 (which is referred as an X-ray source below) configured to scan the object to be scanned in the scanning area with X-ray. Here, the object to be scanned is for example an organism such as a human body, which can include moving objects such as a heart.

The CT image reconstructing device 20 is implemented with for example a general purpose computer or an application-specific integrated circuit, which will be described in detail below. The CT image reconstructing device 306 generates a CT image with a CT image reconstruction such as iterative reconstruction from projection data output by the CT scanner 301.

The CT image output device 30 outputs the CT image reconstructed by the CT image reconstructing device 20. The CT image output device 30 is typically a CT image display device for displaying the CT image output from the CT image reconstructing device 20 on a screen. Of course, the CT image output device 30 is not limited to a CT image display device, and may also be a data transfer interface for transmitting CT images output by CT image reconstructing device 20 via network or a printer for printing CT images output by CT image reconstructing device 20, etc.

The CT images output by the CT system 1 may be used in, for example, a number of fields such as science research and organism data acquisition. In addition, the CT images may also be used as intermediate data for fields like disease diagnosis and health management.

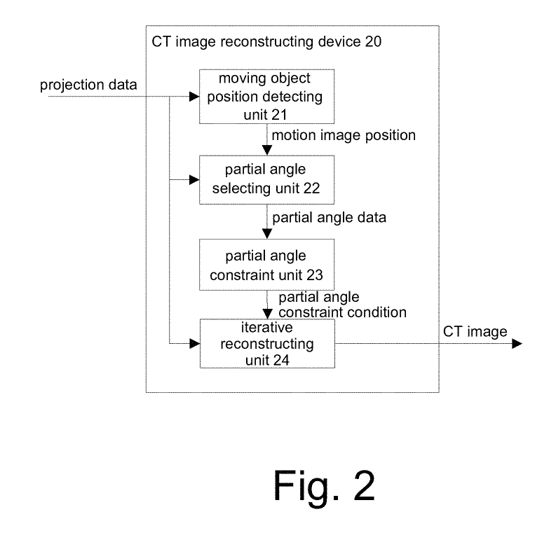

The CT image reconstructing device 20 according to an embodiment of the present invention will be specifically described below. FIG. 2 is a structure block diagram of a CT image reconstructing device according to an embodiment of the present invention. As shown in FIG. 2, a specific structure example of the CT image reconstructing device 20 includes a moving object position detecting unit 21, a partial angle selecting unit 22, a partial angle constrain unit 23 and an iterative reconstructing unit 24. These structures of the CT image reconstructing device 20 may also be implemented with separate circuit structures as hardware, or implemented by a processor executing program stored in memory as function modules.

The moving object position detecting unit 21 detects the basic position of the moving object in the CT image according to the input projection data and outputs the detected motion image position. The partial angle selecting unit 22 selects a view point and an angle range according to the position of moving object and selects data of partial angles in the projection data as the input of the partial angle constrain unit 23 according to the view point and the angle range. The partial angle constrain unit 23 generates a partial angle constrain condition according to the data of partial angles. The iterative reconstructing unit 24 generates a CT image by iterative reconstruction with the partial angle constrain given by the partial angle constrain unit 23 as a constrain item to finally improve temporal resolution of the CT image of the moving object and reduce motion artifacts.

The above-mentioned structure of CT image reconstructing device 20 shown in FIG. 2 is only an example of the CT image reconstructing device of the present invention. It is adequate for the CT image reconstructing device according to the present invention to implement the functions implemented by the above-mentioned structures, without necessarily having the above-described structures.

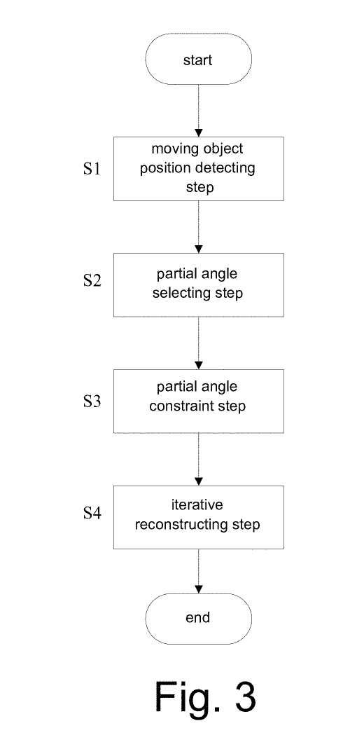

Next, the CT image reconstructing method carried out by the CT image reconstructing device 20 according to an embodiment of the present invention will be specifically described. FIG. 3 is a flow chart of a CT image reconstructing method according to an embodiment of the present invention. As shown in FIG. 3, with the CT image reconstructing method according to an embodiment of the present invention, in a moving object position detecting step S1, the moving object position detecting unit 21 detects position of a moving object in the CT image. Then, in a partial angle selecting step S2, the partial angle selecting unit 22 selects a view point and an angle range according to the position of the moving object and selects data of partial angles in the projection data according to the view point and the angle range. Then, in a partial angle constrain step S3, the partial angle constrain unit 23 generates a partial angle constrain condition according to the data of partial angles. Finally, in an iterative reconstruction step S4, the iterative reconstructing unit 24 generates a CT image by iterative reconstruction with the partial angle constrain condition.

With the CT image reconstructing device 20 and the CT image reconstructing method according to the present embodiment, the partial angle projection data is introduced into the iterative reconstruction algorithm as the constrain condition for data consistency. Since the projection data of partial angles can limit the inconsistency among images during the scanning process, using projection data of partial angles as the constrain condition for the entire restriction result, it is possible to make the iterative reconstruction results proceed toward the direction of meeting consistency, that is, the direction of having less motion artifacts. Therefore, it is possible to improve temporal resolution of the CT image of the moving object and reduce motion artifacts.

A specific example of the CT image reconstructing device and the method thereof according to an embodiment of the present invention will be described below. The specific example is presented only for easily understanding the CT image reconstructing device and the method thereof according to the present embodiment rather than limiting the same. The specific example will be described in detail below with reference to the drawings.

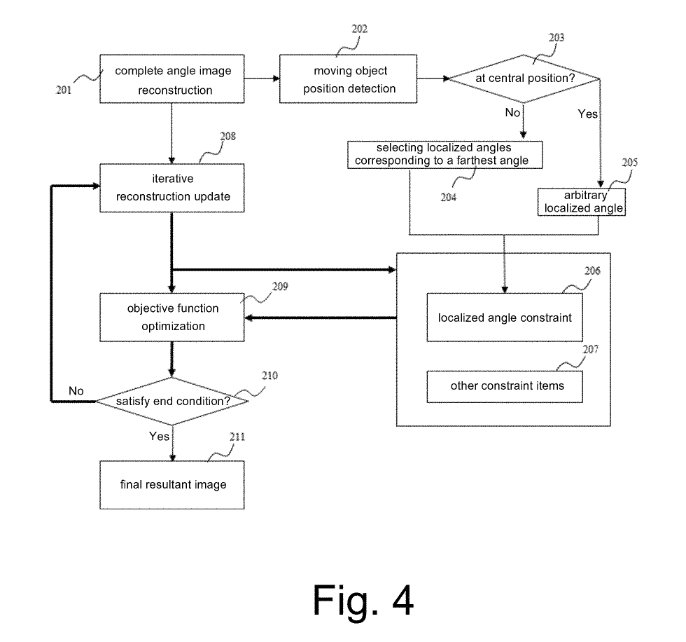

FIG. 4 is a flow chart of a specific example of CT image reconstruction according to an implementation of the present invention. As shown in FIG. 4, in step 201, the CT image reconstructing device 20 firstly implements image reconstruction of complete angle to obtain an initial image that is used as an initial image for iterative reconstruction or used for position detection of the moving object. Image reconstruction of complete angle generally adopts traditional filtered back projection (FBP) which obtains images with the basic reconstruction results but containing many artifacts.

In step 202, the motion image position detecting unit 21 detects motion image position with the initial image obtained in step 201. A specific process will be described in detail hereinblow.

In step 203, after detecting the moving object position in step 202, the partial angle selecting unit 22 determines whether the moving object position belongs to the center region of the CT image. Here, the central region of the CT image refers to a region with a distance from the center of the CT image smaller than a specified threshold. An implementable approach is to calculate the distance from the center point of the moving object to the image center point, and to determine that the moving object's position belongs to the central region of the CT image if the distance between two center points is smaller than the specified threshold. Another implementable approach is to determine whether the region of the moving object covers the center point of the CT image, and determine that the moving object's position belongs to the central region of the CT image if so.

In case that it is determined in step 203 that the moving object's position does not belong to the central region of CT image, step 204 is carried out. In step 204, the partial angle selecting unit 22 selects a view point farthest from the moving object's position and selects data of a preset angle range centered on the selected view point as the data of partial angles (also referred to partial angles herebelow). A specific process will be described in detail hereinblow. Here, the partial angles are not specifically set as long as it's partial angles in the angles of projection data (such as 360 degree). For example, it may be estimated and set as experimental value according to the past history.

In case that it is determined in step 203 that the moving object's position belongs to the central region of CT image, step 205 is carried out. In step 205, the partial angle selecting unit 22 selects an arbitrary view point and selects data of a preset angle range centered on the view point as the data of partial angles. That is, an arbitrary localized angles in the complete angle range of the projection data is selected.

In step 206, the partial angle constrain unit 23 generates a partial angle constraint condition (also referred to as a partial angle constraint herebelow) according to the data of partial angles. The localized angle constraint is for example a sparse constraint item generated in connection with the actual projection data P̅ₗₘₜ and the localized projection data Pₗₘₜ of corresponding angles obtained by forward projection of current image reconstruction results, which may be expressed by formula 5:

Φ₁(P̅ₗₘₜ−Pₗₘₜ) Formula 5

wherein Φ₁ denotes a sparse transformation of the projection data.

In order to be consistent with other image constraint items, the constraint item is transformed into CT image domain, which may be expressed by formula 6.

Ψ₁(x̅ₗₘₜ−Xₗₘₜ) Formula 6

wherein Ψ₁ is the sparse transformation of image data, X̅ₗₘₜ is the image obtained by filtered back projection of the actual localized projection data P̅ₗₘₜ, and Xₗₘₜ is the image obtained by a further filtered back projection of localized projection data Pₗₘₜ of corresponding angles obtained by forward projection of the current image reconstruction results. Thereby, the constraint item of the localized angle projection data is obtained in connection with actual projection data of localized angles and the current image reconstruction results, namely the partial angle constraint condition.

The CT image reconstructing device 20 of the present specific example adds the constraint item of localized angle projection data into the objective function of iterative reconstruction based on a compress sensing theory, and thus the objective function of iterative reconstruction based on the compress sensing theory is expressed by formula 7:

min[α∥₁(X̅ₗₘₜ−Xₗₘₜ)∥₁+(1−α)∥Ψ₂X∥₁] Formula 7

wherein Ψ₂X corresponds to other constraint items generated in step 207. As an example of other constraint items, it may be an entire image constraint condition for the entire image such as a sparse constraint for the image itself in form of TV (total variation) transformation or other forms. α is the weight for weighting the localized angle constraint item proposed in the present invention and other constraint items, with a range of 0˜1, which is typically chosen by experiment according to different application conditions.

In step 208, the iterative reconstructing unit 24 carries out iterative reconstruction update. In the iterative reconstruction update, it is possible to use prior art basic iterative reconstructions such as ART (arithmetic iteration) and SART (Simultaneous Algebraic Reconstruction Technique) etc.

In step 209, the iterative reconstructing unit 24 optimizes the objective function. Common objective function optimization methods such as gradient descent methods may be used for optimizing the objective function.

In step 210, the iterative reconstructing unit 24 determines whether the iteration process satisfies a certain iteration end condition. The iteration constraint condition may be a maximum iteration number or may also be a condition in which a difference between the computer projection of reconstructed image data and the actual projection data is smaller than a certain threshold, or a combination of both.

In case that it is determined in step 210 that the iteration end condition is not satisfied, the process returns to step 208 to continue iterative reconstruction.

In case that it is determined in step 210 that the iteration end condition is satisfied, step 211 is executed. In step 211, the iterative reconstructing unit 24 obtains the resultant final reconstructed image satisfying the constraint condition.

As described above, in the present specific example, in steps S208 to S211, the iterative reconstructing unit 24 generates CT images by iterative reconstruction with the constraint condition obtained by weighted adding of the partial angle constraint condition and the overall image constraint condition.

In the above-mentioned specific example of the present embodiment, step 202 corresponds to the moving object position detection step S1, steps 203 to 205 correspond to the partial angle selection step S2, step 206 corresponds to the partial angle constraint step S3, and steps 208 to 211 correspond to the iterative reconstruction step S4. According to the specific example, the CT image reconstructing device 20 can generate a CT image with less motion artifacts by adding the partial angle constraint condition.

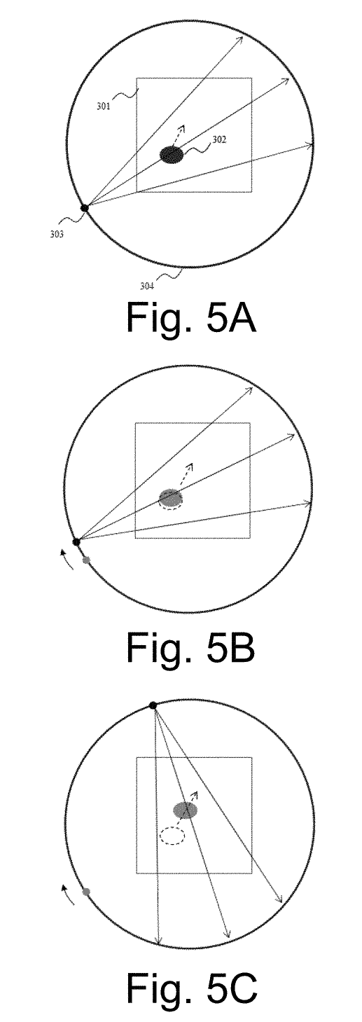

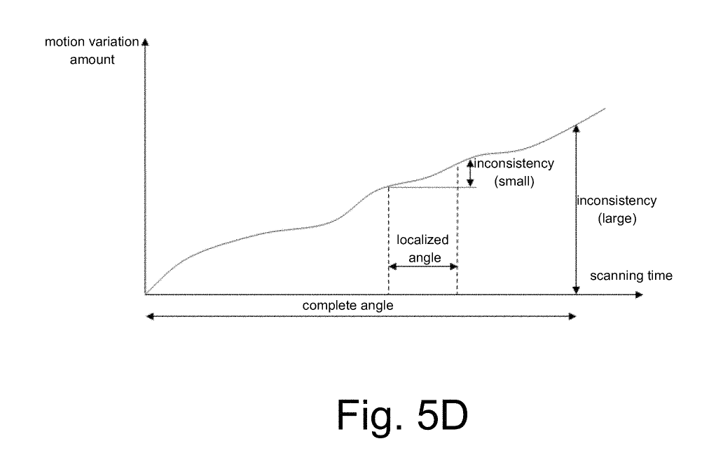

The principle for the present embodiment (and its specific examples) to be able to generate a CT image with less motion artifacts, namely the principle of effectiveness of the partial angle constraint, will be described in detail below. FIGS. 5A to 5D are principle diagrams of the effectiveness of the partial angle constraint according to an embodiment of the present invention. In the figures, 301 is an image, 302 is a moving object, 303 is an X-ray source and 304 is a trajectory of the X-ray source scanning one round. As can be seen from FIGS. 5A, 5B and 5C, when the X-ray source scans a small angle, i.e., scans for a short time (e.g., the case of FIG. 5B), the moving object has less influence on the image consistency, but when the X-ray source scans a large angle, i.e., scans for a long time (e.g., the case of FIG. 5C), the moving object has more influence on the image consistency. This tendency is shown in FIG. 5D. Since the projection data of partial angles can limit the inconsistency among images during the scanning process, using projection data of partial angles as a constrain condition for the entire restriction result, it is possible to make the iterative reconstruction results proceed toward the direction of meeting consistency, that is, the direction of having less motion artifacts.

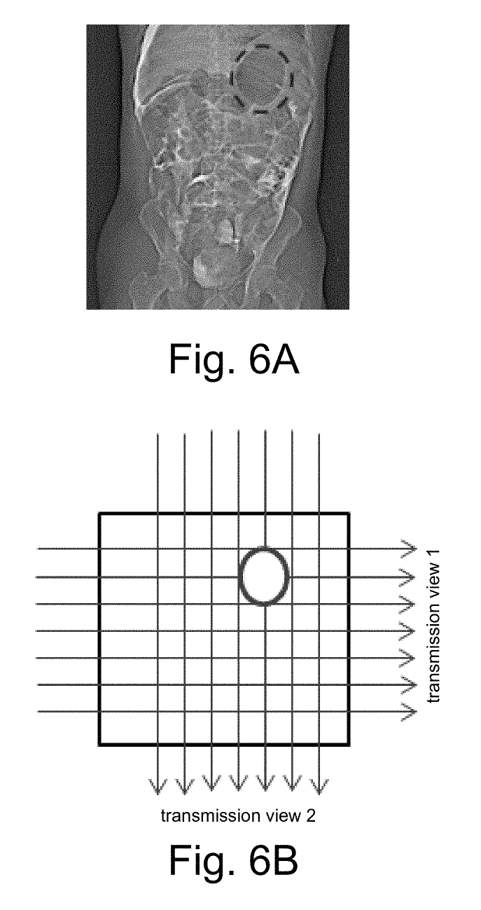

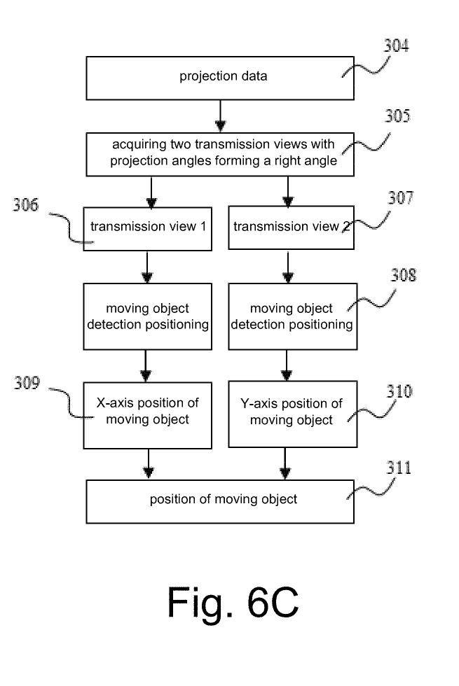

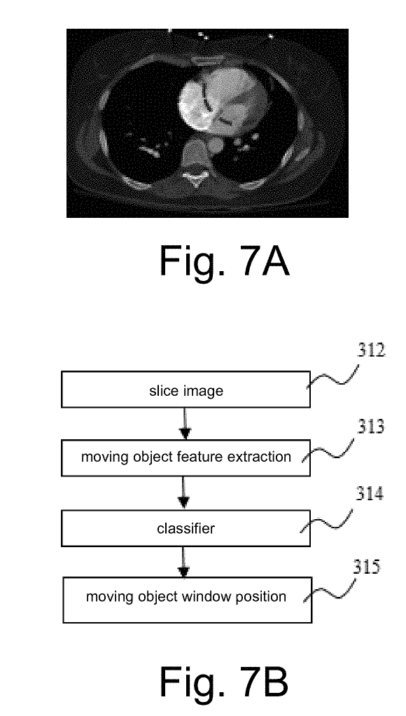

Next, a specific example of detecting moving object's position by the moving object position detecting module 21 in step 202 in the above-mentioned specific example according to the present embodiment. The positioning approach based on a transmission view (FIGS. 6A to 6C) and the positioning approach based on a slice view (typically CT image) (FIGS. 7A and 7B) may be used for the detection of the moving object's position.

FIGS. 6A and 6B are principle diagrams of an approach for detecting a position of a moving object according to a specific example of an embodiment of the present invention, and FIG. 6C is its flow chart. In the positioning approach based on a transmission view, given two transmission views in perpendicular directions, the position coordinates of the moving object on X and Y axes of the CT image are obtained by positioning the moving object in each transmission view. Typically, two perpendicular transmission views just in the X and Y axis directions are chosen according to the coordinate system of the image to facilitate the processing. As shown in the flow chart of FIG. 6C, in step 304, projection data is input. In step 305, two transmission views with projected angles forming a right angle are acquired, thereby obtaining a transmission view 1 and a transmission view 2 in steps 306 and 307. In step 308, detection positioning of the moving object is carried out for the transmission view 1 and the transmission view 2. In steps 309 and 310, X-axis position and Y-axis position of the moving object are determined respectively. Thereby, in step 311, the position of the moving object is detected. The detection for the positioning of the moving object may be an automatic detection method according to the transmission view features of a certain moving object such as heart, or may be determined by the user's input.

FIG. 7A is a principle diagram of another approach for detecting a position of a moving object according to a specific example of an embodiment of the present invention, and FIG. 7B is its flow chart. In the positioning approach based on slice views (typically CT image), as shown in the flow chart of FIG. 7B, in step 312, a slice image is acquired. In step 313, the moving object's features are extracted. In step 314, the classifier for the object is trained. In step 315, the window position of the moving object is detected. The position may be detected in the CT image by training the feature classifier of the moving object using CT image features of some certain moving object such as heart, e.g., shape, texture, and CT value etc. The approach belongs to established technologies in the image detection field and face detection method may be referred to.

In addition, the position detection of the moving object in the present embodiment is not limited to the above-mentioned approach. Various existing detection approaches may be used, or the user may directly specify approximate position for the moving object.

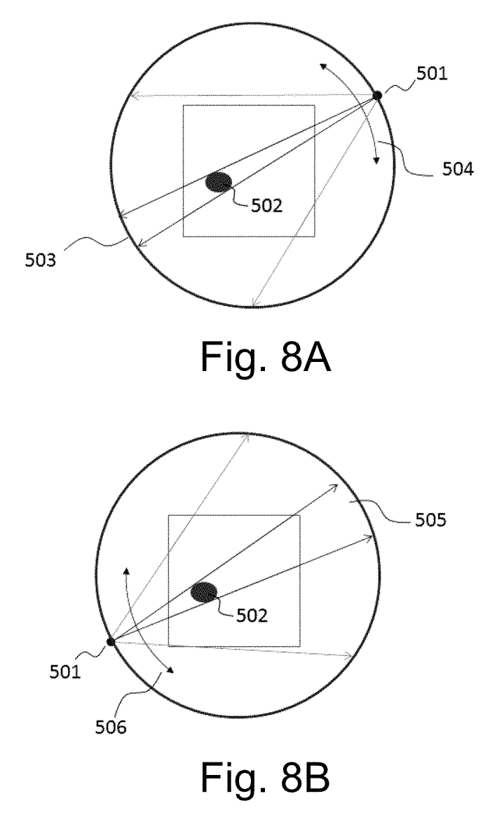

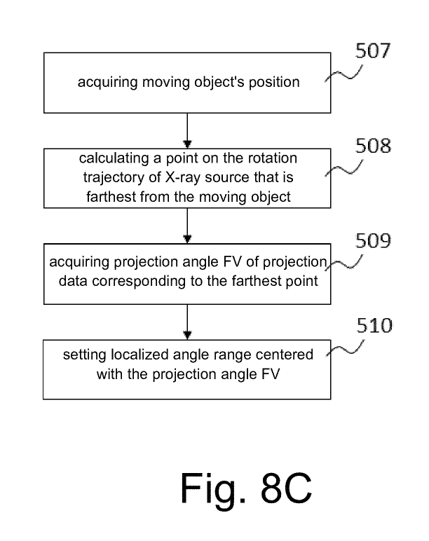

Next, the principle of selecting partial angles by the partial angle selecting module 22 in steps 203 to 205 in the above-mentioned specific example according to the present implementation will be described in detail below. FIGS. 8A and 8B are principle diagrams of partial angle selection and setting according to a specific example of an embodiment of the present invention, and FIG. 8C is its flow chart. In the above-mentioned specific example, the selection of partial angles may be determined according to the position of the moving object. As shown in FIG. 8A, when the X-ray source 501 is on the side far from the moving object 502, the range of influence on projection data by the motion of moving object is as shown by 503. As shown in FIG. 8B, when the X-ray source 501 is on the side nearby the moving object 502, the range of influence on projection data by the motion of moving object is as shown by 505. As can be seen, the range 503 in FIG. 8A is smaller than the range 505 in FIG. 8B. That is, the range of influence on projection data by the motion of moving object when the X-ray source 501 is far from the moving object 502 is larger than when it's nearby the object.

Therefore, in order to have a small influence on projection data by the motion of the moving object, the selection of partial angles shown by 504 in FIG. 8A for which the X-ray source 501 is far from the moving object 502 is superior to selection of partial angles shown by 506 in FIG. 8B for which the X-ray source 501 is nearby the moving object 502. Further, in order to have the X-ray source 501 far from the moving object 502, it is possible to select a view point on a side different from the moving object with respect to the center of the CT image. In the above-mentioned specific example, the view point farthest from the position of the moving object is selected as the view point on the different side from the moving object's position with respect to the center of the CT image.

Therefore, given that the moving object's position does not belong to the central region of the CT image, it is possible to determine the partial angle range as shown in the flow chart of FIG. 8C. In step 507, the partial angle selecting module 22 acquires the moving object's position from the moving object position detecting module 21. In step 508, the partial angle selecting module 22 calculates the point on the rotation trajectory of X-ray source (scanning trajectory) that is farthest from the moving object. In step 509, the partial angle selecting module 22 acquires the projection angle FV of the projection data corresponding to the farthest point. In step 510, the partial angle selecting module 22 sets the range of the partial angles with the projection angle FV as the center. Thereby, by selecting the angle corresponding to the point on the scanning trajectory that is farthest from the moving object as the center point of the partial angles, the partial range of the partial angles may be adjusted as desired, which is set by experiment by trading off the artifact strength and the reconstruction time. Thereby, by setting the angle range according to experimental values with the view point as the center, it is possible to further reduce motion artifacts.

Embodiments of the present invention have been described above with reference to drawings. The above described embodiments are only specific examples of the present invention for understanding the present invention rather than limiting the scope of the present invention. Those skilled in the art can make various modifications, combinations and reasonable omission of elements to embodiments based on the technical spirit of the present invention, and implementations obtained thereby are also included in the scope of the present invention.

1. A CT image reconstructing method for reconstructing a CT image from projection data obtained by X-ray scanning, characterized by comprising:
a moving object position detecting step for detecting a position of a moving object in the CT image;
a partial angle selecting step for selecting a view point and an angle range according to said position of the moving object and selecting data of partial angles in said projection data according to said view point and said angle range;
a partial angle constraint step for generating a partial angle constraint condition according to the data of said partial angles; and
an iterative reconstruction step for generating the CT image by iterative reconstruction with said partial angle constraint condition.

2. The CT image reconstructing method of claim 1, characterized in that,
in case that the position of said moving object does not belong to a central region of the CT image, in said partial angle selecting step, a view point is selected on a side different from the position of said moving object with respect to the center of the CT image;
in case that the position of said moving object belongs to the central region of the CT image, in said partial angle selecting step, an arbitrary view point is selected.

3. The CT image reconstructing method of claim 2, characterized in that,
in case that the position of said moving object does not belong to the central region of the CT image, in said partial angle selecting step, a view point farthest from the position of said moving object is selected.

4. The CT image reconstructing method of claim 1, characterized in that,
in said partial angle selecting step, data of a preset angle range centered on said view point is selected as the data of said partial angles from the projection data.

5. The CT image reconstructing method of claim 1, characterized in that,
in said iterative reconstructing step, the CT image is generated by the iterative reconstruction with a constraint condition obtained by weighted adding of said partial angle constraint condition and an overall image constraint condition.

6. A CT image reconstructing device for reconstructing a CT image from projection data obtained by X-ray scanning, characterized by comprising:
a moving object position detecting unit for detecting a position of a moving object in the CT image;
a partial angle selecting unit for selecting a view point and an angle range according to said position of the moving object and selecting data of partial angles in said projection data according to said view point and said angle range;
a partial angle constraint unit for generating a partial angle constraint condition according to the data of said partial angles; and
an iterative reconstructing unit for generating the CT image by iterative reconstruction with said partial angle constraint condition.

7. The CT image reconstructing device of claim 6, characterized in that,
said partial angle selecting unit is configured to select a view point on a side different from the position of said moving object with respect to a center of the CT image in case that the position of said moving object does not belong to a central region of the CT image;
said partial angle selecting unit is configured to select an arbitrary view point in case that the position of said moving object belongs to the central region of the CT image.

8. The CT image reconstructing device of claim 6, characterized in that,
said partial angle selecting unit is configured to select a view point farthest from the position of said moving object in case that the position of said moving object does not belong to the central region of the CT image.

9. The CT image reconstructing device of claim 6, characterized in that,
said partial angle selecting unit is configured to select data of a preset angle range centered on said view as the data of said partial angles point from the projection data.

10. A CT system for scanning with X-ray and outputting CT images, characterized by comprising:
a CT scanner for scanning with X-ray to obtain projection data;
a CT image reconstructing device for detecting a position of a moving object in a CT image, selecting a view point and an angle range according to said position of the moving object, selecting data of partial angles in said projection data according to said view point and said angle range, generating a partial angle constraint condition according to the data of said partial angles, and generating the CT image by iterative reconstruction with said partial angle constraint condition; and
a CT image output device for outputting the CT image reconstructed by said CT image reconstructing device.
